(19) United States
(12) Patent Application Publication
(10) Pub. No.: US 2024/0177730 A1
(43) Pub. Date: May 30, 2024

(54) INTELLIGENT TRANSCRIPTION AND BIOMARKER ANALYSIS

(71) Applicant: Compass Pathfinder Limited, Altrincham, Cheshire (GB)

(72) Inventors: Robert F. Dougherty, Redwood City, CA (US); Demeter Sztanko, London (GB); Gregory A. Ryslik, Westerville, OH (US); Alexis Harrington, London (GB); Andrew Bettke, Charleston, SC (US)

(21) Appl. No.: 17/927,234

(22) PCT Filed: Nov. 21, 2022
(86) PCT No.: PCT/US22/50603

§ 371 Date: Jun. 1, 2023

Related U.S. Application Data
(60) Provisional application No. 63/282,638, filed on Nov. 23, 2021.
(60) Provisional application No. 63/414,772, filed on Oct. 10, 2022.

(51) Int. Cl. G10L 25/66; G06F 40/47; G10L 15/26; G10L 17/26; G16H 20/70

(52) U.S. Cl. CPC G10L 25/66; G06F 40/47; G10L 15/26; G10L 17/26; G16H 20/70

(57) ABSTRACT

Approaches for transcribing, translating, reviewing, tagging, and providing analytics for therapist sessions are provided. A media file that captures one or more interactions between one or more providers and a recipient of a service may be obtained. A transcript of at least a portion of the one or more interactions captured in the audio file may be generated. Using machine learning, a plurality of analytics may be inferred based, at least in part, upon content contained in the transcript. One or more biomarkers for the recipient may be determined based, at least in part, upon the plurality of analytics. A predicted response to the service to provide for display may be generated based, at least in part, upon the one or more biomarkers and the plurality of analytics.

### CROSS-REFERENCE TO RELATED APPLICATIONS

This application is a 371 of PCT Application No. PCT/US22/50603, entitled “INTELLIGENT TRANSCRIPTION AND BIOMARKER ANALYSIS,” filed on Nov. 21, 2022, which claims priority to U.S. Provisional Patent Application Ser. No. 63/282,638 (APPARATUSES, SYSTEMS, AND METHODS FOR A LANGUAGE AGNOSTIC AI APPROACH TO ENSURE THERAPEUTIC FIDELITY, filed Nov. 23, 2021) and 63/414,772 (INTELLIGENT TRANSCRIPTION AND BIOMARKER ANALYSIS, filed Oct. 10, 2022), which are hereby incorporated herein in their entirety and for all purposes.

### BACKGROUND

Millions of people receive some form of mental health treatment or counseling annually. There have been numerous advancements made in mental health techniques and therapies over time. However, there are still several shortcomings in therapy sessions. For example, during a therapy session, a therapist may attempt to take notes during a session or create their own written records after a session. However, when a therapist is required to take their own notes during a session, the therapist is both breaking concentration and disengaging from the patient. Additionally, when a therapist creates a written record after a session, the record may inadvertently exclude portions of the session due to human recollection. Further, written records may not account for a therapist's or patient's sentiment during a session.

Conventional transcription techniques also may not account for sentiment during a session or provide any insights or analytics about the session. Additionally, human-based transcriptions may introduce errors and manual tagging of therapist sessions may be time-intensive and is not as readily scalable given the number of patients a therapist may see in a given time period and the amount of data a therapist would be required to parse through.

### BRIEF DESCRIPTION OF THE DRAWINGS

Various embodiments in accordance with the present disclosure will be described with reference to the drawings, in which:

FIG. 1 illustrates an example user interface that can be used to implement aspects of the various embodiments.

FIG. 2 illustrates an example audio file waveform that can be utilized to implement aspects of the various embodiments.

FIG. 3 illustrates example analytics that can be extracted, generated, or computed in accordance with various embodiments.

FIG. 4 illustrates an example system that can be used to implement aspects of the various embodiments.

FIG. 5 illustrates an example method that can be used to implement one or more aspects of the various embodiments.

FIG. 6 illustrates an example of an environment for implementing aspects in accordance with various embodiments.

FIG. 7 illustrates an example block diagram of an electronic device that can be utilized to implement one or more aspects of the various embodiments.

FIG. 8 illustrates components of another example environment in which aspects of various embodiments can be implemented.

### DETAILED DESCRIPTION

Approaches for transcribing, translating, reviewing, tagging, and providing analytics for therapist sessions are provided. One or more interactions between one or more providers and a recipient of a service may be recorded and automatically transcribed according to Natural Language Processing (NLP) techniques or Artificial Intelligence techniques. Interactions may include patient interactions with their medical care team, including, but not limited to, interactions between a patient and a therapist or interactions between a patient and a general practitioner, among other such options. In accordance with an example embodiment, individual utterances may have timestamps associated with the utterances, indicating where an utterance may lie in the audio file or the transcript. The timestamps may be associated with speaker identifiers. In some embodiments, confidence scores indicative of how confident a system is at determining the time stamp may be provided. Further, a session type, length of recording, location, and date of recording may be provided, among other such options. Through use of the system, a rich database of transcription and automated analytics may be generated, allowing the NLP algorithm and/or other artificial intelligence techniques to be utilized for automated tagging and/or biomarker discovery. Analytics may be provided in near-real time or real time.

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

As used herein, “artificial intelligence” may include, but is not limited to, machine learning, natural language processing, neural networks, random forest models, and other such algorithms or models.

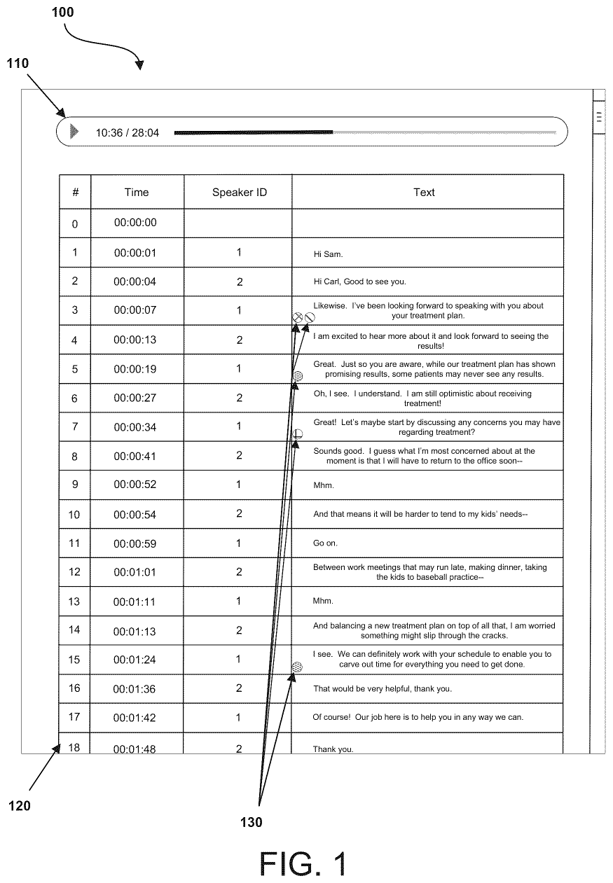

FIG. 1 illustrates an example user interface 100 that can be used to implement aspects of the various embodiments. As shown, an example interface 100 may include features to control audio 110. The audio may be audio from a media file that was taken during a therapist session. In some embodiments, a therapist session may occur before, during, and/or after an initial treatment, such as a dosing session with psilocybin for treatment of a medical condition. A table 120 may also be provided for display. The table 120 may include utterances as provided under the “#” column, time stamps as provided under the “Time” column, Speaker IDs as provided under the “Speaker ID” column, and transcribed text corresponding to the media file, as shown under the “Text” column. One or more indicators 130 may also be provided. In an example embodiment, the indicators 130 may correspond to tags which may identify a indications associated with the therapy session, or biomarkers of the patient and one or more corresponding features of the patient that enables the system or user to find metabiomarkers of the person. For example, a patient's affective state, which may be a biomarker in at least some embodiments, may be used to train a tagging model. A machine learning model may utilize the tagged data and generate one or more new biomarkers that a user may not have initially considered or disclosed.

In at least one embodiment, the tagging system may be provided for display as an interactive table, enabling a user to apply one or more tags to one or more strings of text. The interactive table may display a time at which corresponding text was spoken, the individual who spoke (based on identifiers), and the content of the speech. In at least one embodiment, the user may be presented with a pop-up window or another screen, enabling the user to select one or more tags of a plurality of tags. In an example embodiment, tags may include indications such as “therapist discusses grounding techniques,” or “therapist discusses external support,” among other such options. Individual tags may be displayed with associated colors or other indicators such as symbols. If a user chooses to assign tags to a string of text, the string of text may be displayed, in accordance with an example embodiment, with the colors and/or indicators that correlate to the selected tags. In embodiments where video recordings of a session have been recorded, video data may be tagged or labeled using computer vision techniques.

In some embodiments, tagging may be automated such that a set of training data, comprising tagged items, may be utilized with a machine learning algorithm to associate pieces of text from a transcript with a tag. A trained machine learning model may predict one or more tags of transcripts. In at least some example embodiments, the predicted tags may be provided for display.

In accordance with an example embodiment, NLP and AI techniques may be utilized to identify one or more biomarkers indicative of a patient's progress in their therapy journey and/or flag the biomarkers for a therapist's review. In an example embodiment, a biomarker may correspond to any digitally identifiable signal of voice, speech pattern, tonality, frequency/pitch, sentiment, syntax, diction, or any sensory input including, but not limited to, audio, visual, textual, or tactile input. In some example embodiments, the biomarkers may be provided for display. Biomarkers may be generated, in accordance with an example embodiment, based on one or more data inputs, or they may be manually entered by a therapist or administrator. Machine learning may be utilized, in accordance with an example embodiment, to analyze sensor data and determine if a pattern can be detected to predict an outcome of interest. Supervised or unsupervised learning approaches may be utilized.

Data inputs may account for sensor data, including data received through one or more smart phones, tablets, cellular phones, touchscreen devices, smart watches, personal computers, accelerometers, gyroscopes, respiration sensors, body movement sensors, proximity sensors, motion sensors, ambient light sensors, moisture sensors, temperature sensors, compasses, barometers, fingerprint sensors, facial identification sensors, cameras, pulse sensors, heart rate variability (HRV) sensors, beats per minute (BPM) heart rate sensors, microphones or other sound sensors, speakers, and GPS or other location sensors. In at least some embodiments, multiple sensors may be utilized to collect a combination of data. In some embodiments, multiple sensors may be communicatively coupled.

In an example embodiment, a patient may have a therapy session with a therapist. Such a session may occur shortly after (e.g., one day or one week) after an initial treatment session, such as after a dosing session using psilocybin for treatment of a health condition. This example is not intended to be limiting, and one or more aspects of the various embodiments may also be utilized and/or performed prior to or during a therapy treatment session. In this example embodiment, the therapist session may be recorded and transcribed, translated, and/or annotated. The therapy session may be analyzed in an automated fashion to determine whether a patient or therapist has adhered to a pre-determined therapeutic model.

In an example embodiment, the therapeutic session may be translated from one language to another. For example, a text-based representation of the session may be maintained in any language. In some embodiments, a session may be transcribed prior to translation. In other embodiments, a session may be transcribed after translation.

One or more aspects of the present embodiment may be provided through a web application. In accordance with one or more embodiments, a user may manually rate and tag a therapy session. Additionally, a user may view automatically rated and tagged sessions, such as sessions that a computing system analyzes and tags. For example, a computing system may be configured to apply Natural Language Processing (NLP) and/or artificial intelligence (AI) techniques to transcribe, translate, review, rate, and/or flag items of interest from one or more sessions and/or therapists. NLP may be utilized, in accordance with an example embodiment, to automatically generate a transcription of the session. Using AI, one or more biomarkers related to a patient's progress may be identified and flagged for a therapist's review. AI may be configured to improve the understanding of mechanisms of action and/or mechanisms of change for a particular therapy. For example, AI may be utilized to predict how a patient is responding to psilocybin therapy, or determine how a patient may be feeling during a preclinical stage of their therapy. A biomarker may be any digitally identifiable signal of voice, speech pattern, tonality, frequency/pitch, syntax, diction, or any sensory input including, but not limited to, audio, visual, textual, or tactile input. Biomarkers may be generated based on a data input, such as sensor data. In some embodiments, biomarkers may be manually entered by a therapist and/or administrator. In an example embodiment, biomarkers may be determined and evaluated at pre-determined times. For example, biomarkers may be analyzed prior to treatment, during treatment, and/or after treatment. Additionally, the system may predict patient suitability for a type of treatment. For example, the system may determine a suitability score for a patient based on various factors, including if a patient asked more questions and therefore may be more open to the experience prior to treatment, or whether/how much a patient has reviewed literature related to the treatment. A suitability score may be provided for display to a therapist via a mobile application or web-based application, among other such options.

In NLP, an “utterance” may be defined as a spoken group of words that is preceded by and followed by a pause. In contrast, a “sentence” may refer to a group of words that express a complete thought. According to one or more embodiments described herein, an “utterance” may be an amalgamation of the two. For example, if a sentence occurs over several utterances, the utterances may be combined to form a single utterance. Alternatively, if an utterance contains several sentences, each sentence may be extracted and can be treated as stand-alone utterances.

After using NLP to transcribe an audio file, individual utterances within the transcript may be determined. For example, a language model may be utilized to parse the transcript into individual utterances. The language model may consider factors such as punctuation or terms which may relate to the same topic within a given period of time. Additional processing steps may be taken after transcribing. For example, Unicode characters may be converted to ASCII characters. Redacted information such as names may be replaced with non-personally identifiable alternatives (e.g., Jane/John Doe). According to another example embodiment, transcriber comments may be replaced with model-familiar text. For example, a transcription note “[LAUGHING]” may be replaced with “haha” so that the model may better-recognize the term and use the term in generating scores.

Sentiment analysis of a piece of text may include scoring the text as being positive or negative. Text may be scored in two dimensions: valence (e.g., positivity) and arousal (e.g., energy or activation). In this way, sentiment may capture intensity, rather than just positivity or negativity. Sentiment score of a piece of text may be distinguishable from trying to infer an emotional state of a speaker. For example, text reciting “I love broccoli” may be scored by traditional sentiment models as being positive. However, if vocalized in a sarcastic way, the text would signal a negative attitude towards broccoli. Additionally, text reciting “I love broccoli” may be ranked as 97% positive in one model, and text reciting “I like broccoli” may be ranked as 98% positive in the same model. The similarity in positivity score may be a result of the sentiment analysis problem being treated as a classification problem, and not considering intensity. In such cases, the model may only care about whether the “positive” or “negative” label is correct. In accordance with one or more embodiments described herein, for individual utterances, a classifier may be used to score the likelihood that the utterance should belong to one of the following four classes: “happy,” “angry,” “gloomy,” and “calm.” While this example embodiment describes these four classes, other classes indicative of sentiment may be utilized. For a given utterance, the model may provide scores for individual classes. To determine a session sentiment score, a set of utterances of a given speaker (e.g., participant or patient, therapist, etc.) during a single visit (e.g., first preparation, first integration, etc.) may be given a probability measure, where the measure of a given utterance is proportional to a number of words in the utterance. From this, mean valence and arousal scores may be obtained. The scores may be provided for display along with a transcription or as a separate page, in at least one example embodiment.

In at least one example embodiment, a therapy session may be recorded via one or more sensors, such as microphones. In an example embodiment, a microphone may be arranged such that it results in at least two different audio tracks, allowing for spatial source separation for various users within the session. The recorded audio may be stored, such as to a local device comprising memory capable of being coupled to a computing device, or stored directly to a computing device. In other embodiments, the recorded audio may be uploaded to cloud-based storage. Prior to transcription, audio may be pre-processed to reduce background noise. Pre-processing may also include ensuring individual tracks are dedicated to a single speaker. Examples of such pre-processing may include, but are not limited to, blind source separation or winner-takes-all sound audio filtering, among other such options.

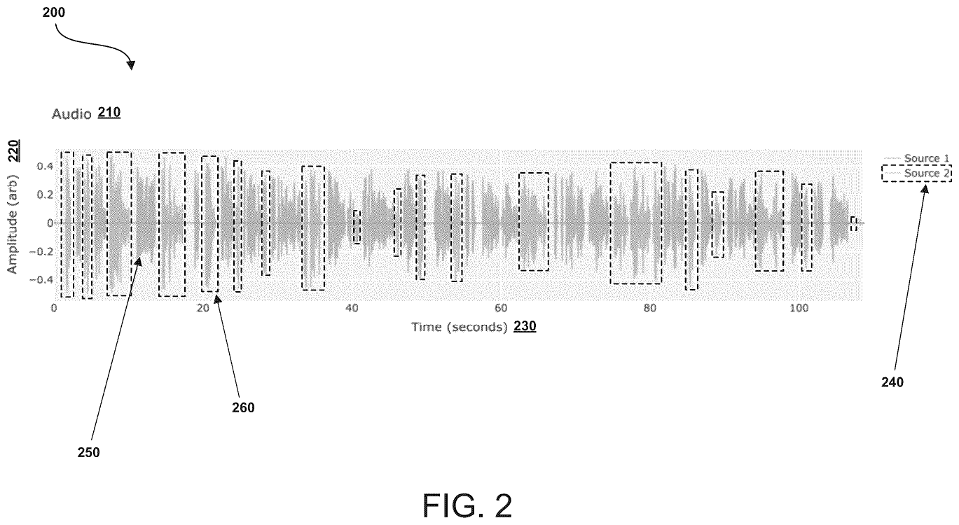

In accordance with an example embodiment, a visualization of the audio may be provided, such as in the form of illustrated sound waves. FIG. 2 illustrates an example audio file waveform 200 that can be utilized to implement aspects of the various embodiments. In this example, audio from the therapist and audio from the patient 240 may be presented in an easily identifiable format, such as by presenting individual waveforms 250, 260 in different colors or line formats. The waveforms in this example embodiment may be provided as amplitude 220 over time 230. The waveforms may be utilized, in an example embodiment, to determine one or more biomarkers pertaining to how a patient or therapist may have been feeling during a session. A user may also be presented with an option of isolating and/or independently adjusting the volume of each party. For example, the user may decrease the volume of the therapist to better hear the patient. Further, the volumes of both the therapist and patient may be adjusted and/or normalized automatically.

In some example embodiments, transcription may occur on the fly, in real time while the therapy session is occurring. Transcription may convert the audio file to a text file (for example, a JSON or similar format). The transcription may note or link to one or more time points related to time points in the audio file. Transcription, in accordance with an example embodiment, may include neural net transcription and translation, such that the neural net is utilized to generate the transcript and/or generate a translation of the session recording. In some embodiments, a therapy session may be video recorded, and biomarkers may be collected based on detected visual characteristics such as facial expressions and body language interpreted through a computing system.

In some embodiments, a therapy session may occur in person or via a computing device such as a device having a camera and associated microphone. In other embodiments, a therapy session may occur through use of a virtual reality device.

A transcript may be uploaded to a web application or mobile application for a therapist or reviewer to see. Additionally, the audio file may be uploaded so that the therapist or reviewer can listen and make any corrections to the file. Corrections may also, in accordance with an example embodiment, be automatically flagged by a system based on one or more predictions. For example, the system may detect that parts of the transcript could not be transcribed with sufficient confidence (e.g., a threshold level of confidence), indicating that those parts of the transcript may not have been accurately transcribed. Based on those flags, the system may predict alternative transcriptions that a therapist or reviewer may select from, or the therapist/reviewer may listen to the audio file at various flagged time points and manually correct the transcription. For example, a user may have stated “I was feeling . . . um . . . well this morning,” but the system may have determined that the user could have said “um . . . well” or “unwell.” Such a discrepancy may result in a low confidence value, causing the system to raise this as an issue. By flagging this potential discrepancy to a therapist or reviewer, the transcript may have higher overall fidelity because a corrected transcript may more accurately represent how a patient was feeling during the therapist session. In some example embodiments, the system may automatically perform corrections to the transcript based on determined alternatives. For example, a machine learning model may utilize training data to analyze a transcript and predict one or more corrections to the transcript. In accordance with one or more embodiments, corrections may be applied to utterances that may have been incorrectly split from the transcript.

Tagging may be utilized, in accordance with an example embodiment, to provide analytics of fidelity and predicted patient outcomes, as well as for NLP and AI analysis of potential digital biomarkers. For example, tagging may involve prompting a user to ask or respond to follow-up questions related to a corresponding piece of text. For example, if a sleep disturbance is identified or noted in a user's data, a follow-up question may be asked about the user's sleep. Further, tagging may be utilized to request a therapist to adhere to a specific part of a therapeutic model. Tagging may additionally provide a user with information which may be relevant to an identified portion of text. Such questions and information may be configured to aid a user in categorizing, rating, reviewing, and/or tagging portions of text. Additionally, tagging may be utilized to determine and ensure that a therapist has adhered to a specific therapeutic model and/or performed all the necessary critical points for the therapy session. In this way, fidelity within the session can be ensured, which is an important aspect of especially sensitive therapy sessions such as a first session after an initial dosing treatment. Analytics may be provided in near-real time or real time. This example is not intended to be limiting, and one or more aspects of the various embodiments may also be utilized and/or performed prior to or during a therapy treatment session.

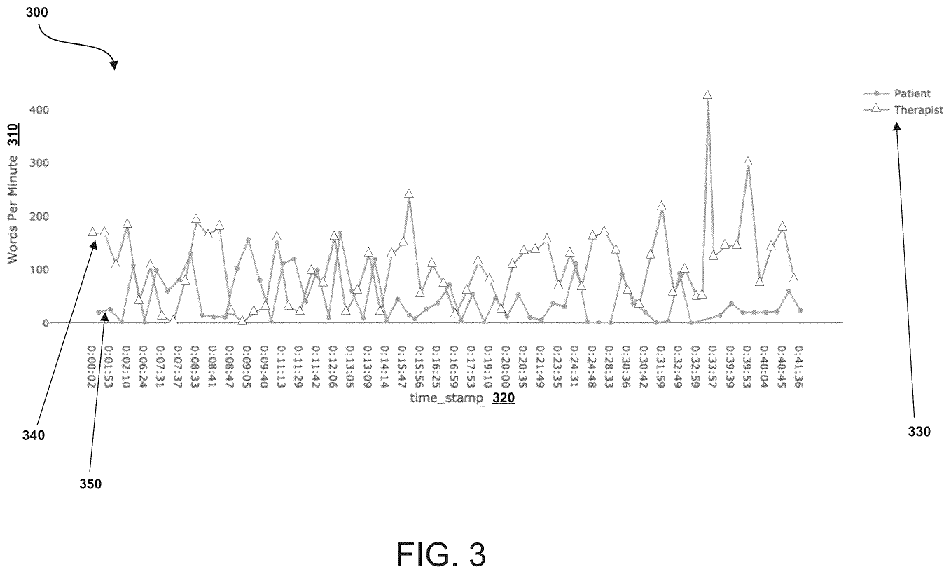

FIG. 3 illustrates example analytics 300 that can be extracted, generated, or otherwise computed in accordance with various embodiments. This example embodiment is being illustrated with respect to a specific analysis of words per minute over time, and is not intended to be limiting. Computed analytics may be processed using one or more techniques, including mathematical models, statistical models, and computer science analysis including, but not limited to, dimensionality reduction, clustering, NLP, and/or generalized linear modeling. Machine learning techniques could be utilized, in accordance with an example embodiment, to help predict one or more therapeutic outcomes associated with a user. Analytics may be provided in a dashboard format to one or more users, such as authenticated users, for review. In accordance with an example embodiment, AI may be utilized to identify potential language biomarkers. The system may consider various analytics including, but not limited to, pitch/frequency, words per minute 310, frequency of pauses, and/or duration of pauses for individual speakers 330 over time 320. The analytics may illustrate, as shown in FIG. 3, analytics associated with the individual speakers as a graph 340, 350. Further, the analytics may be categorized based on when the data was collected. For example, the system may compute analytics before, during, and/or after administration of a treatment, and both sets of analytics may be provided for display. The system may extract differences between the sets of data and provide visual indicators suggesting potential explanations or other insights for the differences. For example, using machine learning, the system may generate insights based on differences between pre- and post-dosing. In another example embodiment, dynamic support for a therapist may be provided for display in real time or quasi-real time, to aid the therapist in making clinical decisions and improving patient care. Such support may be generated using artificial intelligence. As a non-limiting example, when a therapist is meeting with a patient, a machine learning model may prompt areas for exploration or areas of a therapeutic model that were not robustly covered in a prior session. Alternatively, real time feedback could be provided to a therapist to prompt the therapist to cover required content if the system detects that the content was not covered in a previous timeframe of the session.

Analytics, in an example embodiment, may include a plurality of metrics. For example, an audio recording may be analyzed for speaking speed, including the patient's speed and optionally the therapist's speed. Speed may be measured in words per minute, or any other suitable unit. The system may present a graph representing the speaking speed, such as in the form of “words per minute vs. time points.” A patient's speaking speed and the therapist's speaking speed may be presented as different color line graphs, or by other visual indicators.

In an example embodiment, tags may include both a biomarker of the patient and one or more corresponding features of the patient that enables the system or user to find metabiomarkers of the person. For example, a patient's affective state, which may be a biomarker in at least some embodiments, may be used to train the tagging model. A machine learning model may utilize the tagged data and generate one or more new biomarkers that a user may not have initially considered or disclosed.

In at least one embodiment, the tagging system may be provided for display as an interactive table, enabling a user to apply one or more tags to one or more strings of text. The interactive table may display a time at which corresponding text was spoken, the individual who spoke (based on identifiers), and the content of the speech. In at least one embodiment, the user may be presented with a pop-up window or another screen, enabling the user to select one or more tags of a plurality of tags. In an example embodiment, tags may include indications such as “therapist discusses grounding techniques,” or “therapist discusses external support,” among other such options. Individual tags may be displayed with associated colors or other indicators such as symbols. If a user chooses to assign tags to a string of text, the string of text may be displayed, in accordance with an example embodiment, with the colors and/or indicators that correlate to the selected tags. In embodiments where video recordings of a session have been recorded, video data may be tagged or labeled using computer vision techniques. For example, using computer vision techniques, biomarkers may be labeled at a given timepoint and provided for display. In this example, video data of a patient may be analyzed using machine learning, and digital biomarkers may be extracted. In this embodiment, digital biomarkers may include, but are not limited to, physical agitation, facial expressions, blush response, and eye contact, among other such biomarkers. Tabs or filters may be provided within the interactive table such that a particular category or subset of information may be displayed. Tabs or filters, in accordance with an example embodiment, may include but are not limited to “transcribed,” “anonymized,” “edited,” and “labeled.”

In addition to tagging, other features may be provided for display including, but not limited to, commenting, tagging other users to specific modules within the service, highlighting sections for review, and providing spaces for collaborative notes.

In accordance with an example embodiment, a user may select a specific string of text. One or more parameters, including but not limited to the user's selection and various identifiers associated with the session, may be sent to a backend database. Accordingly, the user selection may be verified and passed along to the database. In such an embodiment, tags may be sorted and/or filtered algorithmically, either deterministically or by using artificial intelligence. The tags may be sorted and/or filtered to provide the most appropriate tag for the user selection based on NLP or AI and one or more of the parameters. Sorted tags may be returned to the front-end web application/mobile application, where they may be provided for display to the user.

The tagging system and machine learning model may be General Data Protection Regulation (GDPR) compliant and Health Insurance Portability and Accountability Act (HIPAA) compliant. For example, an audio file may be streamed, but not copied by, a user. Further, users may be assigned specific roles with associated access permissions. Users may also be assigned identification numbers so that personal information such as names is not compromised. The system may utilize data from any number of patients without disclosing the identity and/or source of the data. In this way, machine learning models utilized in the system may train on collective patient data without compromising a patient's personal identifying information. Further, any steps performed on the data may be captured and logged with a version history. Additionally, the process for a user to sign in may be user-specific with policies in place to help ensure data is protected. In some example embodiments, verified users may be provided with the option to utilize a quick response (QR) code or other embedded identifier to sign in and access allowed features.

In accordance with an example embodiment, principal component analysis (PCA) may be employed when analyzing the session recordings. PCA may be utilized to visualize the data set. For example, patterns or variations between data sets may be visualized using PCA. Patterns may include the number of words used, the types of words used, or other related factors. A two-dimensional PCA graph may be provided for presentation to a user, where data from individual speakers may be color-coded or otherwise provided to indicate the speaker.

In at least one embodiment, the system may calculate an average speed duration of individual speakers during a session. The system may further compare the average duration of speech by each individual in relation to an average duration of speech of other parties (e.g., in cases of different patient and therapist pairings). The system may present the user with mean durations of the parties' speech according to the therapist's identifier. The system may also provide for display a visual representation of the actual speech durations for both parties according to a particular session.

In some example embodiments, a user may be able to review and rate the fidelity of a given session. For example, a user may be provided with a plurality of prompts, sorted by category, enabling a user to rate the completeness of each prompt. Categories in this example may include, but are not limited to, preparation, dosing, and integration. One example prompt may be “Whether the therapist covered relevant information about the treatment.” If the user determines that the therapist did not cover relevant information about the treatment, then the user may enter an answer such as “Absent.” In some embodiments, a user may respond to prompts using a slider bar or other such interface. Additionally, the user may be provided with the option to include their own notes after each prompt. The responses for the prompts may be aggregated to determine an overall fidelity rating. Further, the responses of each prompt of each category may be aggregated to determine category-specific fidelity ratings.

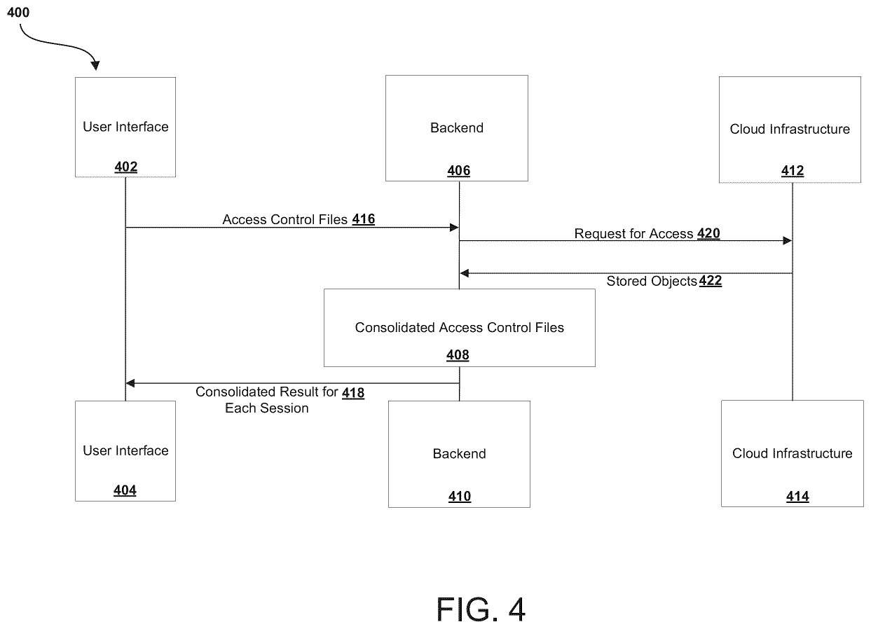

FIG. 4 illustrates an example system 400 that can be used to implement aspects of the various embodiments. In this example embodiment, a user may be provided with a tagging feature which may be presented to the user as an interactive table through one or more user interfaces 402, 404, enabling the user to apply one or more tags to strings of text. Prior to seeing the interactive table, one or more access control files 416 may be sent and verified at one or more backend servers 406, 410 to ensure that the user is able to view the content. In response to a request for access 420, the user may have access to view the content through cloud infrastructure 412, 414. Stored objects may be returned 422, and updated or consolidated access control files 408 may be stored. The consolidated result may be provided 418 to a user interface 402, 404. The interactive table may display the time at which the text was spoken, the individual who spoke, and the content of the speech, among other such options. In an example embodiment, the user may be presented with a pop-up window or another screen, enabling the user to pick from a list of a plurality of tags. The interactive table may include a number of tabs and/or filters which may be configured to display a category or set of text strings.

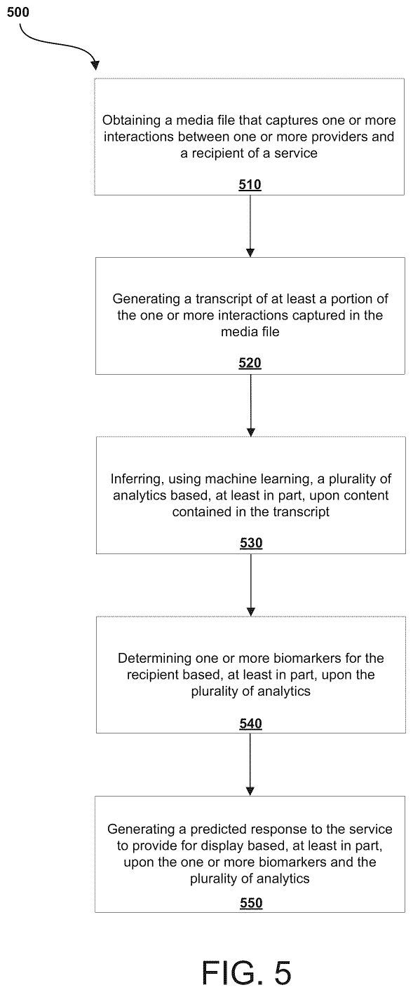

FIG. 5 illustrates an example method 500 that can be used to implement one or more aspects of the various embodiments. In accordance with one or more embodiments, a media file capturing one or more interactions between one or more providers and a recipient of a service may be obtained 510. In accordance with an example embodiment, the one or more providers may be a member of a medical care team providing a healthcare service, and the recipient may be a patient of the service. A transcript of at least a portion of the one or more interactions captured in the audio file may be generated 520. Using a neural network, a plurality of analytics may be inferred based, at least in part, upon content contained in the transcript 530. One or more biomarkers for the patient may be determined based, at least in part, upon the plurality of analytics 540. A predicted response to the service to provide for display may be generated based, at least in part, upon the one or more biomarkers and the analytics 550.

It should be understood that for any process herein there can be additional, fewer, or alternative steps performed in similar or alternative orders, or in parallel, within the scope of the various embodiments unless otherwise specifically stated.

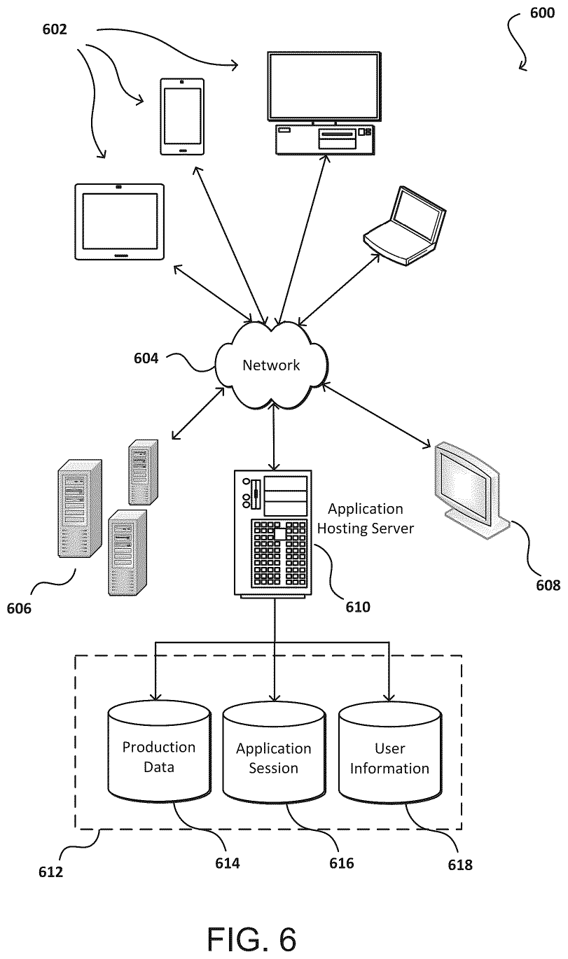

As discussed, different approaches can be implemented in various environments in accordance with the described embodiments. For example, FIG. 6 illustrates an example of an environment 600 for implementing aspects in accordance with various embodiments. As will be appreciated, although a Web-based environment is used for purposes of explanation, different environments may be used, as appropriate, to implement various embodiments. The system includes an electronic client device 602, 608, which can include any appropriate device operable to send and receive requests, messages or information over an appropriate network 604 and convey information back to a user of the device. Examples of such client devices include personal computers, cell phones, handheld messaging devices, laptop computers, set-top boxes, personal data assistants, electronic book readers and the like. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the environment includes one or more servers 606 for receiving requests and serving content in response thereto, although for other networks, an alternative device serving a similar purpose could be used, as would be apparent to one of ordinary skill in the art.

The illustrative environment includes at least one application server 610 and a data store 612. It should be understood that there can be several application servers, layers or other elements, processes or components, which may be chained or otherwise configured, which can interact to perform tasks such as obtaining data from an appropriate data store. As used herein, the term “data store” refers to any device or combination of devices capable of storing, accessing and retrieving data, which may include any combination and number of data servers, databases, data storage devices and data storage media, in any standard, distributed or clustered environment. The application server 610 can include any appropriate hardware and software for integrating with the data store 612 as needed to execute aspects of one or more applications for the client device and handling a majority of the data access and business logic for an application. The application server provides access control services in cooperation with the data store and is able to generate content such as text, graphics, audio and/or video to be transferred to the user, which may be served to the user by the one or more servers 606, including a Web server, in the form of HTML, XML or another appropriate structured language in this example. The handling of all requests and responses, as well as the delivery of content between the client device 602, 608 and the application server 610, can be handled by the Web server of servers 606. It should be understood that the Web and application servers are not required and are merely example components, as structured code discussed herein can be executed on any appropriate device or host machine as discussed elsewhere herein.

The data store 612 can include several separate data tables, databases or other data storage mechanisms and media for storing data relating to a particular aspect. For example, the data store illustrated includes mechanisms for storing production data 614 and user information 618, which can be used to serve content for the production side. The data store is also shown to include a mechanism for storing log or application session data 616. It should be understood that there can be many other aspects that may need to be stored in the data store, such as page image information and access rights information, which can be stored in any of the above listed mechanisms as appropriate or in additional mechanisms in the data store 612. The data store 612 is operable, through logic associated therewith, to receive instructions from the application server 610 and obtain, update or otherwise process data in response thereto. In one example, a user might submit a request for transcribing, tagging, and/or labeling a media file. In this case, the data store might access the user information to verify the identity of the user and can provide a transcript including tags and/or labels along with analytics associated with the media file. The information can then be returned to the user, such as in a results listing on a Web page that the user is able to view via a browser on the user device 602, 608. Information for a particular feature of interest can be viewed in a dedicated page or window of the browser.

Each server typically will include an operating system that provides executable program instructions for the general administration and operation of that server and typically will include computer-readable medium storing instructions that, when executed by a processor of the server, allow the server to perform its intended functions. Suitable implementations for the operating system and general functionality of the servers are known or commercially available and are readily implemented by persons having ordinary skill in the art, particularly in light of the disclosure herein.

The environment in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 6. Thus, the depiction of the system 600 in FIG. 6 should be taken as being illustrative in nature and not limiting to the scope of the disclosure.

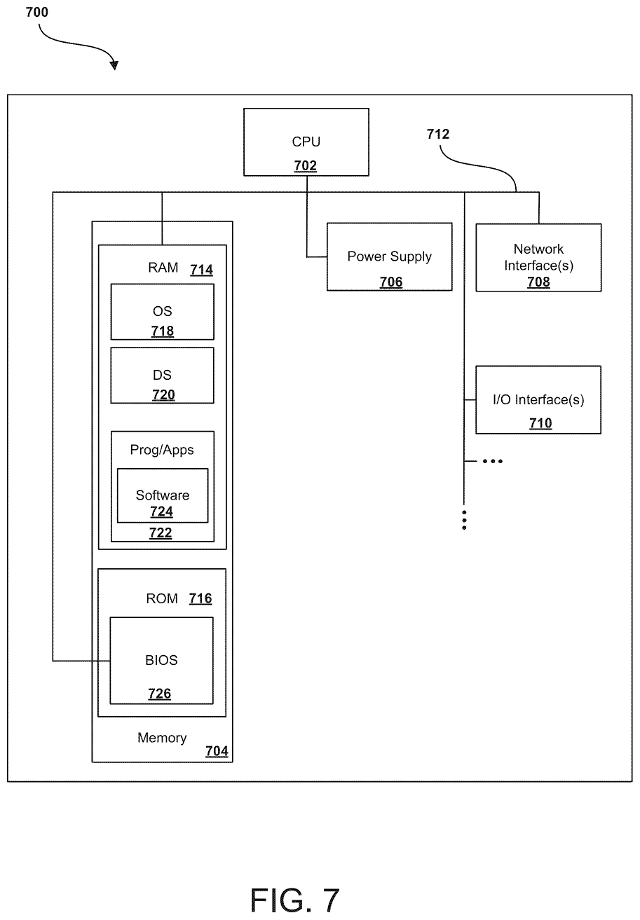

FIG. 7 illustrates an example block diagram of an electronic device that can be utilized to implement one or more aspects of the various embodiments. Instances of the electronic device 700 may include one or more servers and one or more client devices. In general, the electronic device may include a processor/CPU 702, memory 704, a power supply 706, and input/output (I/O) components/devices 710, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 700. A touchscreen may determine whether a user is providing input by, for example, determining whether the user the user is touching the touchscreen with a part of the user's body such as their fingers. The electronic device 700 can also include a communications bus 712 that connects to the aforementioned elements of the electronic device 700. Network interfaces 708 can include a receiver and a transmitter (or a transceiver), and one or more antennas for wireless communications.

The processor 702 can include one or more of any type of processing device, e.g., a Central Processing Unit (CPU), and a Graphics Processing Unit (GPU). Also, for example, the processor can utilize central processing logic, or other logic, may include hardware, firmware, software or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 704, which can include Random Access Memory (RAM) 714 and Read Only Memory (ROM) 716, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 718, data storage 720, which may include one or more databases, and programs and/or applications 722, which can include, for example, software aspects of the program 724. The ROM 716 can also include Basic Input/Output System (BIOS) 726 of the electronic device 700.

Software aspects of the program 722 are intended to broadly include or represent all programming, applications, algorithms, models, software and other tools necessary to implement or facilitate methods and systems according to embodiments of the invention. The elements may exist on a single computer or be distributed among multiple computers, servers, devices, or entities.

The power supply 706 may contain one or more power components, and may help facilitate supply and management of power to the electronic device 700.

The input/output components, including Input/Output (I/O) interfaces 710, can include, for example, any interfaces for facilitating communication between any components of the electronic device 700, components of external devices, and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 710 and the bus 712 can facilitate communication between components of the electronic device 700, and in an example can ease processing performed by the processor 702.

Where the electronic device 700 is a server, it can include a computing device that can be capable of sending or receiving signals, e.g., a wired or wireless network, or may be capable of processing or storing signals, e.g., in memory as physical memory states. The server may be an application server that includes a configuration to provide one or more applications via a network to another device. Also, an application server may, for example, host a website that can provide a user interface for administration of example embodiments.

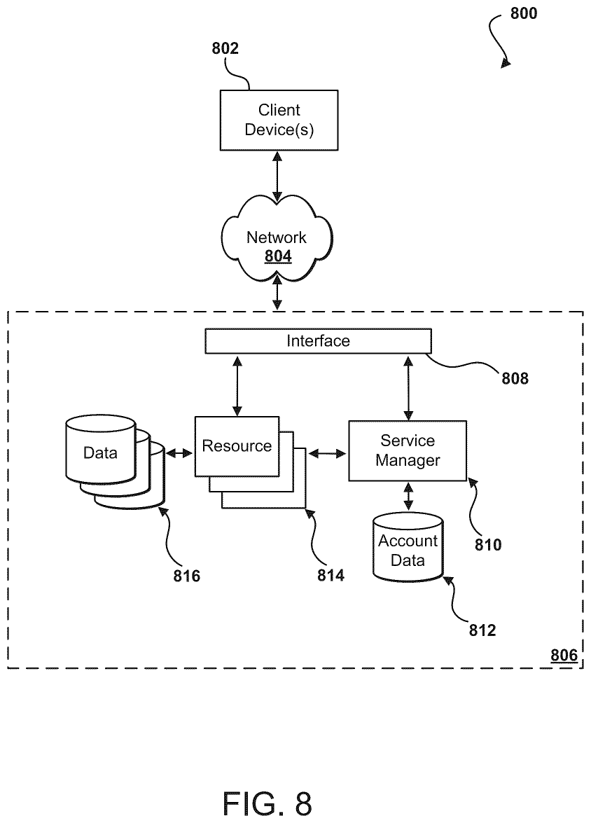

FIG. 8 illustrates an example environment 800 in which aspects of the various embodiments can be implemented. In this example a user is able to utilize one or more client devices 802 to submit requests across at least one network 804 to a multi-tenant resource provider environment 806. The client device can include any appropriate electronic device operable to send and receive requests, messages, or other such information over an appropriate network and convey information back to a user of the device. Examples of such client devices include personal computers, tablet computers, smart phones, notebook computers, and the like. The at least one network 804 can include any appropriate network, including an intranet, the Internet, a cellular network, a local area network (LAN), or any other such network or combination, and communication over the network can be enabled via wired and/or wireless connections. The resource provider environment 806 can include any appropriate components for receiving requests and returning information or performing actions in response to those requests. As an example, the provider environment might include Web servers and/or application servers for receiving and processing requests, then returning data, Web pages, video, audio, or other such content or information in response to the request.

In various embodiments, the provider environment may include various types of resources that can be utilized by multiple users for a variety of different purposes. As used herein, computing and other electronic resources utilized in a network environment can be referred to as “network resources.” These can include, for example, servers, databases, load balancers, routers, and the like, which can perform tasks such as to receive, transmit, and/or process data and/or executable instructions. In at least some embodiments, all or a portion of a given resource or set of resources might be allocated to a particular user or allocated for a particular task, for at least a determined period of time. The sharing of these multi-tenant resources from a provider environment is often referred to as resource sharing, Web services, or “cloud computing,” among other such terms and depending upon the specific environment and/or implementation. In this example the provider environment includes a plurality of resources 814 of one or more types. These types can include, for example, application servers operable to process instructions provided by a user or database servers operable to process data stored in one or more data stores 816 in response to a user request. As known for such purposes, the user can also reserve at least a portion of the data storage in a given data store. Methods for enabling a user to reserve various resources and resource instances are well known in the art, such that detailed description of the entire process, and explanation of all possible components, will not be discussed in detail herein.

In at least some embodiments, a user wanting to utilize a portion of the resources 814 can submit a request that is received to an interface layer 808 of the provider environment 806. The interface layer can include application programming interfaces (APIs) or other exposed interfaces enabling a user to submit requests to the provider environment. The interface layer 808 in this example can also include other components as well, such as at least one Web server, routing components, load balancers, and the like. When a request to provision a resource is received to the interface layer 808, information for the request can be directed to a service manager 810 or other such system, service, or component configured to manage user accounts and information, resource provisioning and usage, and other such aspects. A service manager 810 receiving the request can perform tasks such as to authenticate an identity of the user submitting the request, as well as to determine whether that user has an existing account with the resource provider, where the account data may be stored in at least one data store 812 in the provider environment. A user can provide any of various types of credentials in order to authenticate an identity of the user to the provider. These credentials can include, for example, a username and password pair, biometric data, a digital signature, a QR-based credential, or other such information.

The provider can validate this information against information stored for the user. If the user has an account with the appropriate permissions, status, etc., the resource manager can determine whether there are adequate resources available to suit the user's request, and if so can provision the resources or otherwise grant access to the corresponding portion of those resources for use by the user for an amount specified by the request. This amount can include, for example, capacity to process a single request or perform a single task, a specified period of time, or a recurring/renewable period, among other such values. If the user does not have a valid account with the provider, the user account does not enable access to the type of resources specified in the request, or another such reason is preventing the user from obtaining access to such resources, a communication can be sent to the user to enable the user to create or modify an account, or change the resources specified in the request, among other such options. In at least some example embodiments, a user may be authenticated to access an entire fleet of services provided within a service provider environment. In other example embodiments, a user's access may be restricted to specific services within the service provider environment using one or more access policies tied to the user's credential(s).

Once the user is authenticated, the account verified, and the resources allocated, the user can utilize the allocated resource(s) for the specified capacity, amount of data transfer, period of time, or other such value. In at least some embodiments, a user might provide a session token or other such credentials with subsequent requests in order to enable those requests to be processed on that user session. The user can receive a resource identifier, specific address, or other such information that can enable the client device 802 to communicate with an allocated resource without having to communicate with the service manager 810, at least until such time as a relevant aspect of the user account changes, the user is no longer granted access to the resource, or another such aspect changes.

The service manager 810 (or another such system or service) in this example can also function as a virtual layer of hardware and software components that handles control functions in addition to management actions, as may include provisioning, scaling, replication, etc. The resource manager can utilize dedicated APIs in the interface layer 808, where each API can be provided to receive requests for at least one specific action to be performed with respect to the data environment, such as to provision, scale, clone, or hibernate an instance. Upon receiving a request to one of the APIs, a Web services portion of the interface layer can parse or otherwise analyze the request to determine the steps or actions needed to act on or process the call. For example, a Web service call might be received that includes a request to create a data repository.

An interface layer 808 in at least one embodiment includes a scalable set of user-facing servers that can provide the various APIs and return the appropriate responses based on the API specifications. The interface layer also can include at least one API service layer that in one embodiment consists of stateless, replicated servers which process the externally-facing user APIs. The interface layer can be responsible for Web service front end features such as authenticating users based on credentials, authorizing the user, throttling user requests to the API servers, validating user input, and marshalling or unmarshalling requests and responses. The API layer also can be responsible for reading and writing database configuration data to/from the administration data store, in response to the API calls. In many embodiments, the Web services layer and/or API service layer will be the only externally visible component, or the only component that is visible to, and accessible by, users of the control service. The servers of the Web services layer can be stateless and scaled horizontally as known in the art. API servers, as well as the persistent data store, can be spread across multiple data centers in a region, for example, such that the servers are resilient to single data center failures.

The various embodiments can be further implemented in a wide variety of operating environments, which in some cases can include one or more user computers or computing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system can also include a number of workstations running any of a variety of commercially-available operating systems and other known applications for purposes such as development and database management. These devices can also include other electronic devices, such as dummy terminals, thin-clients, gaming systems and other devices capable of communicating via a network.

Most embodiments utilize at least one network that would be familiar to those skilled in the art for supporting communications using any of a variety of commercially available protocols, such as TCP/IP, FTP, UPnP, NFS, and CIFS. The network can be, for example, a local area network, a wide-area network, a virtual private network, the Internet, an intranet, an extranet, a public switched telephone network, an infrared network, a wireless network and any combination thereof. In embodiments utilizing a Web server, the Web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more Web applications that may be implemented as one or more scripts or programs written in any programming language, such as Java®, C, C # or C++ or any scripting language, such as Perl, Python or TCL, as well as combinations thereof. The server(s) may also include database servers, including without limitation those commercially available from Oracle®, Microsoft®, Sybase® and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, optical storage devices and solid-state storage devices such as random access memory (RAM) or read-only memory (ROM), as well as removable media devices, memory cards, flash cards, etc. Such devices can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information.

The system and various devices also typically will include a number of software applications, modules, services or other elements located within at least one working memory device, including an operating system and application programs such as a client application or Web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed. Storage media and other non-transitory computer readable media for containing code, or portions of code, can include any appropriate media known or used in the art, such as but not limited to volatile and non-volatile, removable and non-removable media implemented in any method or technology for storage of information such as computer readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

What is claimed is:

1. A computer-implemented method, comprising:
obtaining an audio file that captures one or more interactions between one or more providers and a recipient of a service;
generating a transcript of at least a portion of the one or more interactions captured in the audio file;
inferring, using machine learning, a plurality of analytics based, at least in part, upon content contained in the transcript;
determining one or more biomarkers for the recipient based, at least in part, upon the plurality of analytics; and
generating a predicted response to the service to provide for display based, at least in part, upon the one or more biomarkers and the plurality of analytics.

2. The computer-implemented method of claim 1, wherein the recording is transcribed using Natural Language Processing (NLP).

3. The computer-implemented method of claim 1, further comprising:
detecting that the transcript contains an error;
providing an indication of the error; and
suggesting one or more corrections to the error.

4. The computer-implemented method of claim 1, further comprising:
analyzing one or more utterances present in the transcript;
generating one or more tags associated with the one or more utterances; and
inferring the plurality of analytics based, at least in part, upon the generated tags.

5. The computer-implemented method of claim 1, wherein the one or more biomarkers are determined based, at least in part, upon at least one of: detected sentiment, a detected pitch, a detected frequency, determined words per minute, detected pauses, and a duration of pauses in the audio file.

6. The computer-implemented method of claim 1, further comprising:
assigning one or more labels to the audio file based, at least in part, upon audio cues detected in the audio file.

7. A system comprising:
at least one processor; and
at least one memory, storing instructions that, when executed by the at least one processor, cause the at least one processor to:
obtain a media file that captures one or more interactions between one or more providers and a recipient of a service;
generating a transcript of at least a portion of the one or more interactions captured in the media file;
infer, using machine learning, a plurality of analytics based, at least in part, upon content contained in the transcript;
determine one or more biomarkers for the recipient based, at least in part, upon the plurality of analytics; and
generate a predicted response to the service to provide for display based, at least in part, upon the one or more biomarkers and the plurality of analytics.

8. The system of claim 7, wherein the instructions that, when executed by the at least one processor, cause the at least one processor to further:
detect that the transcript contains an error;
provide an indication of the error; and
suggest one or more corrections to the error.

9. The system of claim 7, wherein the recording is transcribed using Natural Language Processing (NLP).

10. The system of claim 7, wherein the instructions that, when executed by the at least one processor, cause the at least one processor to further:
analyze one or more utterances present in the transcript;
generate one or more tags associated with the one or more utterances; and
infer the plurality of analytics based, at least in part, upon the generated tags.

11. The system of claim 7, wherein the one or more biomarkers are determined based, at least in part, upon at least one of: detected sentiment, a detected pitch, a detected frequency, determined words per minute, detected pauses, and a duration of pauses in the media file.

12. The system of claim 7, wherein the instructions that, when executed by the at least one processor, cause the at least one processor to further:
assign one or more labels to the media file based, at least in part, upon audio or visual cues detected in the media file.

13. The system of claim 7, wherein the media file is pre-processed prior to transcription to filter out unwanted noise from the media file.

14. A non-transitory computer-readable medium, storing instructions that, when executed by at least one processor, cause the at least one processor to:
obtain a media file that captures one or more interactions between one or more providers and a recipient of a service;
generating a transcript of at least a portion of the one or more interactions captured in the media file;
infer, using machine learning, a plurality of analytics based, at least in part, upon content contained in the transcript;
determine one or more biomarkers for the recipient based, at least in part, upon the plurality of analytics; and
generate a predicted response to the service to provide for display based, at least in part, upon the one or more biomarkers and the plurality of analytics.

15. The non-transitory computer-readable medium of claim 14, wherein the instructions that, when executed by the at least one processor, cause the at least one processor to further:
detect that the transcript contains an error;
provide an indication of the error; and
suggest one or more corrections to the error.

16. The non-transitory computer-readable medium of claim 14, wherein the recording is transcribed using Natural Language Processing (NLP).

17. The non-transitory computer-readable medium of claim 14, wherein the instructions that, when executed by the at least one processor, cause the at least one processor to further:
analyze one or more utterances present in the transcript;
generate one or more tags associated with the one or more utterances; and
infer the plurality of analytics based, at least in part, upon the generated tags.

18. The non-transitory computer-readable medium of claim 14, wherein the one or more biomarkers are determined based, at least in part, upon at least one of: detected sentiment, a detected pitch, a detected frequency, determined words per minute, detected pauses, and a duration of pauses in the media file.

19. The non-transitory computer-readable medium of claim 14, wherein the instructions that, when executed by the at least one processor, cause the at least one processor to further:
assign one or more labels to the media file based, at least in part, upon audio or visual cues detected in the media file.

20. The non-transitory computer-readable medium of claim 14, wherein the media file is pre-processed prior to transcription to filter out unwanted noise from the media file.
